(19) United States
(12) Patent Application Publication
(10) Pub. No.: US 2009/0265812 A1
(43) Pub. Date: Oct. 22, 2009

(54) Plant Gene Promoter and its Use

(75) Inventors: David Granot, Jerusalem (IL); David Granot, Jerusalem (IL); Dvora Swartzberg, Petach-Tikva (IL); Marcelo German, Newark, DE (US)

Correspondence Address: MARTIN D. MOYNIHAN d/b/a PRTSI, INC., P.O. BOX 16446, ARLINGTON, VA, 22215, US

(73) Assignee: The State of Israel,Ministry of Agriculture& Rural Development, Agricultural Research Organization, Beit-Dagan (IL)

(21) Appl. No.: 12/225,881

(22) PCT Filed: Mar. 29, 2007
(86) PCT No.: PCT/IL2007/000407

§ 371 Date: Apr. 24, 2009

Related U.S. Application Data
(60) Provisional application No. 60/790,777, filed on Apr. 11, 2006.

(51) Int. Cl. A01H 5/00; C12N 15/82; C07H 21/04; C12N 5/04

(52) U.S. Cl. 800/289; 800/298; 800/278; 536/23.1; 435/320.1; 435/419

(57) ABSTRACT

A method for protecting a plant from high temperature stress (HTS), comprising transforming the plant with a polynucleotide sequence. The polynucleotide sequence comprises a nucleotide sequence encoding a sugar kinase under the control of an anther and/or pollen specific promoter, thereby producing a transformed plant having improved tolerance to HTS. Also disclosed is the polynucleotide sequence of the LeFRK4 promoter.

### FIELD OF THE INVENTION

This invention relates to a plant gene promoter and its use.

### REFERENCES

The following references are referred to in the specification and are identified in the text by their respective numbers. Mention of the reference does not necessarily indicate that it is relevant to the patentability of the invention.
1. Imin N, Kerim T, Rolfe B G, Weinman J J (2004) Effect of early cold stress on the maturation of rice anthers. Proteomics 4: 1873-1882;
2. Aloni B, Peet M, Pharr M, Karni L (2001) The effect of high temperature and high atmospheric CO2 on carbohydrate changes in bell pepper (Capsicum annuum) pollen in relation to its germination. Physiol Plant 112: 505-512;
3. Pressman E, Peet M M, Pharr D M (2002) The effect of heat stress on tomato pollen characteristics is associated with changes in carbohydrate concentration in the developing anthers. Ann Bot (Lond) 90: 631-636;
4. German M A, Asher I, Petreikov M, Dai N, Schaffer A A, Granot D (2004) Cloning, expression and characterization of LeFRK3, the fourth tomato (Lycopersicon esculentum Mill.) gene encoding fructokinase. Plant Science 166: 285-291
5. Kandel-Kfir M, Damari-Weissler H, German M A, Gidoni D, Mett A, Belausov E, Petreikov M, Adir N, and Granot D (2006). Two newly identified membrane-associated and plastidic tomato HXKs: characteristics, predicted structure and intracellular localization. Planta 224, 1341-1352.
6. German M A, Dai N, Chmelnitsky I, Sobolev I, Salts Y, Barg R, Schaffer A A, Granot D (2002) LeFRK4, a novel tomato (Lycopersicon esculentum Mill.) fructokinase specifically expressed in stamens. Plant Science 163: 607-613;
7. Karni, L and Aloni, B (2002) Fructokinase and hexokinase from pollen grains of bell pepper (Capsicum annuum L.): possible role in pollen germination under conditions of high temperature and CO2 enrichment; Annals of Botany, v. 90 (5):607-612;
8. Dai N, Schaffer A, Petreikov M, Shahak Y, Giller Y, Ratner K, Levine A, Granot D (1999) Overexpression of Arabidopsis hexokinase in tomato plants inhibits growth, reduces photosynthesis, and induces rapid senescence. Plant Cell 11: 1253-1266.

### BACKGROUND OF THE INVENTION

The main factor responsible for the reduction of tomato crop yields under elevated temperatures is the impairment of pollen development and germination. This factor is known as high temperature stress (HTS). The flowering phases most sensitive to high temperatures are meiosis (8-9 days before anthesis) and fertilization (1-3 days after anthesis). In Brassica, pollen from plants exposed to four days of HTS (35° C.) had lower in vitro germination rates (17.5%) than pollen from control grown plants (59.2%). The lower germination rate was regardless of whether in vitro germination was carried out at 23° C. or 35° C. Similar findings indicating that the male reproductive organs are the most sensitive to heat stress were reported for other plant species as well.

Although male sterility due to HTS has been observed in numerous plants, the biochemical or developmental pathways affected remain unknown. Similarly, while much is known about the response of vegetative tissues to environmental stress, very little information is available with respect to the pollen's ability to mount a defensive response. A comparative analysis of the global pollen protein expression profile in the presence or absence of stress is one approach that can help address these shortcomings. To date, there are still very few comprehensive analyses specific to the pollen proteome and only one such analysis deals with temperature stress [1]. In this study the global protein expression profile of rice anthers was analyzed under cold stress and 70 protein spots were found to be differentially displayed. The little evidence so far seems to indicate that pollen grains not only do not induce the proteins normally found in stressed vegetative tissues, but that the detrimental effects occur mainly at the translational and post-translational level.

Carbohydrates play a critical role in pollen development, germination and fertilization, serving as nutrients for metabolic, structural, storage and perhaps signaling functions. In many plants including tomato and Brassica, the transported sugar is mainly sucrose. An unloading pathway of sucrose via the apoplastic space is mandatory for symplastically isolated cells, such as developing pollen, that receive their carbohydrate supply from the tapetum layer and the surrounding locular fluid. The transported sucrose is released from the sieve elements of the phloem into the anther wall layers and the tapetum apoplast, probably via a sucrose transporter. The released sucrose molecules may follow either or both of the following routes: 1. Enter the cytosol of the pollen sink cells and be cleaved by sucrose synthase (SuSy) into fructose and UDP-glucose (UDPG); 2. Irreversibly hydrolyzed into glucose and fructose by an extra-cellular (apoplastic) invertase—ionically bound to the cell wall. The resulting hexose monomers, glucose and fructose, are then taken up into the sink cells by hexose transporters.

Pollen germination and tube growth are also highly dependent on the availability of sugars, since the main metabolic activity during this process is the biosynthesis of polymers that will form the elongating cell wall. While the volume of the protoplast does not change significantly during pollen tube elongation since it moves forward, the cell wall forms an immobile tube which continuously expands at the apex. Given that a single pollen tube has to achieve a total length of about a centimeter in tomato and Brassica pistils within a short time, the supply of cell wall precursors needs to be uninterrupted and extremely rapid. To support this high level of carbohydrate synthesis pollen tubes use both stored reserves which can include sucrose, starch, phytic acid and lipids, depending on the species and external sources of sugar. In in vitro growing pollen tubes, the need for external carbon sources was shown to increase with time as internal stores were depleted. Efficient sugar metabolism is therefore crucial for the success of the fertilization process.

In a number of plant species, stress-induced impairment of male gametophyte development is preceded by disturbances in carbohydrate metabolism in the anther and the developing pollen [2]. In tomato, HTS causes a reduction in both starch levels of developing immature tomato pollen and soluble sugar concentration of mature pollen grains. This was accompanied by a decrease in the number of pollen grains produced, an elevated number of non-viable pollen and a decrease in the capacity of the viable pollen to germinate [3].

Both sucrose synthase (SuSy) and invertase cleave sucrose to produce the hexose sugars, glucose and fructose, that must be phosphorylated by hexose phosphorylating enzymes (hexose kinases) before they can be further metabolized. Hexose phosphorylating enzymes are characterized by their particular affinity to various sugars. Hexokinase (HXK) phosphorylates glucose and fructose, whereas fructokinase (FRK) phosphorylates only fructose. The affinity of FRKs to fructose are one to two orders of magnitude higher than that of HXKs to fructose. The phosphorylated sugars (hexose phosphates) may enter glycolysis or the pentose phosphate pathway, or be converted and stored as starch. Uridine diphosphate glucose (UDPG), produced by cleavage of sucrose by SuSy, may also be used for cell wall, cellulose or starch synthesis.

Four HXK genes and four FRK genes (LeFRK1-4) were identified in tomato plants [4,5]. All four HXK and three of the FRK genes (LeFRK1-3) are expressed at different levels in all plant tissues [4,5]. However, the fourth recently cloned fructokinase, LeFRK4, is expressed exclusively in anthers and pollen [6] (mainly in mature and germinating pollen but also in developing pollen) at levels 100 times higher than any of the other HXK and FRK genes.

Karni, L. and Aloni, B. [7] investigated changes in fructokinase (FRK) and hexokinase activities in pepper flowers during their development, and studied the possible roles of these enzymes in determining pollen germination capacity under high temperature and under CO₂ enrichment, previously shown to modify sugar concentrations in pepper pollen. Their results suggested that pollen and anther FRK may play a role in the regulation of pollen germination, possibly by providing fructose-6-phosphate for glycolysis, or through conversion to UDPG to support the biosynthesis of cell wall material for pollen tube growth.

### SUMMARY OF THE INVENTION

In one aspect of the invention, there is provided an isolated polynucleotide sequence comprising one of the following: (a) the LeFRK4 promoter; and (b) a functional part thereof, being a modified LeFRK4 promoter in which one or more of the nucleotide bases have been substituted or deleted, or one or more nucleotide bases have been added thereto, wherein the specific promoter activity of the modified promoter is substantially the same as that of the LeFRK4 promoter.

The term “LeFRK4 promoter” includes the sequence consisting of nucleotides 1 to 2488 (SEQ. ID. NO:1) of the sequence shown in FIG. 6. In a preferred embodiment, the term refers to nucleotides 1175 to 2488 (SEQ. ID. NO: 2) of the sequence shown in FIG. 6. It should be noted that the 14 bp sequence at the beginning of the LeFRK4 gene (bp 2489-2502 in FIG. 6) is apparently not expressed, and may be a 5′ UTR sequence. The promoter nucleotide sequence 1175 to 2488 (SEQ. ID. NO: 2) has been deposited at GenBank on Mar. 19, 2006, and has been accorded accession number DQ453118.

The term “specific promoter activity” within the context of the LeFRK4 promoter includes at least the following features:
1. the ability of the promoter to drive the expression of a gene, preferably a gene which may encode a sugar kinase, and in particular, hexokinase and/or fructokinase;
2. the promoter is active primarily, and preferably exclusively, in the anther and pollen portions of the plant.

The term “substantially the same” within the context of a modified LeFRK4 promoter means that the modified promoter has a specific promoter activity having at least one, and preferably both of its features, defined above, at a level of at least 80%, more preferably at least 85%, still more preferably at least 90%, even more preferably at least 95%, most preferably at least 98% of the activity of the unmodified, native promoter.

Also included in the invention is a vector, preferably an expression vector, comprising the promoter of the invention. Various expression vectors active in plant cells are well known to the skilled man of the art, e.g. PVX, pJLX, pBI121, pART7/27, pRT104, etc. Various heterologous nucleic acid sequences or genes may be inserted into the vector so as to be operably linked to and under control of the promoter. In a preferred embodiment, the heterologous gene encodes a protein involved in sugar metabolism such as an invertase, a sucrose synthase or a glucose transporter. In a most preferred embodiment, the heterologous gene encodes a sugar kinase, for example hexokinase, fructokinase, fucokinase or galactokinase (GalK).

The heterologous gene may also be a variable gene having a special purpose such as a reporter gene confirming the expression of the promoter. In the case of producing a male sterile plant, the foreign gene may be a gene degrading the development or germination of the pollen.

A further embodiment of the invention includes a host cell transformed by the vector of the invention. In a preferred embodiment, the host cell is a plant cell, preferably a pollen or an anther cell. A still further embodiment of the invention is a transgenic plant comprising the host cell of the invention.

The promoter of the invention may be used in various applications in plant genetics. One of the applications is described in detail below.

Other possible applications are: (1) causing male sterility by specific expression of various genes (such as RNAses, proteases, toxins or other genes) that may disrupt pollen or anther development. Male sterility is a desirable trait for the production of hybrid seeds and may save laborious sterilization methods currently used in company seeds; and (2) elimination of pollen development to prevent or reduce allergenic pollen components or allergic effects caused by pollen.

In a second aspect of the invention, there is provided a method for protecting a plant from high temperature stress (HTS), comprising transforming the plant with a polynucleotide sequence, wherein said polynucleotide sequence comprises a nucleotide sequence encoding a sugar kinase under the control of an anther and/or pollen specific promoter, thereby producing a transformed plant having improved tolerance to HTS.

In a preferred embodiment, the specific promoter comprises the LeFRK4 promoter. In another preferred embodiment, the sugar kinase is a hexokinase or a fructokinase. In a still other preferred embodiment, the plant is tomato, cucumber, peppers, zucchini, maize, cotton, flax, wheat, rice, eggplant, melon or Brassica, or any other plant susceptible to HTS.

In a third aspect of the invention, there is provided a method for causing male sterility comprising transforming the plant with a polynucleotide sequence, wherein said polynucleotide sequence comprises a nucleotide sequence capable of disrupting pollen or anther development under the control of an anther and/or pollen specific promoter, thereby causing male sterility. In a preferred embodiment, the specific promoter comprises the LeFRK4 promoter.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a bar graph showing the effect of HTS on pollen development and germination. Pollen of tomato plants grown for several weeks at either normal (28° C./22° C.-day/night) or high (32/26° C.) temperatures were collected and germinated at low (23° C.) and high (32° C.) temp. Pollen viability was scored under the microscope following staining with Alexander reagent as described in Pressman E, Moshkovitch H, Rosenfeld K, Shaked R, Gamliel B and Aloni B (1998), Influence of low night temperatures on sweet pepper flower quality and the effect of repeated pollinations, with viable pollen, on fruit setting. Journal of Horticultural Science and Biotechnology 73: 131-136. Viable pollen includes germinating and stained grains;

FIG. 2 is a bar graph showing expression of 35S::AtHXK1 in anther and pollen. The expression of the Arabidopsis hexokinase AtHXK1 in tomato plants under 35S promoter was determined in mature pollen, anther (without pollen) and germinating pollen. The expression was determined by real time PCR with AtHXK1 specific primers, and normalized to the expression of a house keeping gene cyclophilin (similar results were obtained upon normalization with rRNA);

FIG. 3 includes photographs and bar graphs and shows the effect of HTS on pollen germination. Control tomato plants (MP, w.t.) and independent isogenic transgenic tomato lines overexpressing AtHXK1 (HK37, HK4 and HK38) were grown for several weeks at either normal (A) (28° C./22° C.-day/night) or high (B) (32/26° C.) temperatures. HK4 and HK38 plants had high expression of AtHXK1 whereas HK37 plants had a moderate expression level [8]. Pollen were collected and germinated at low (23° C.) and high (32° C.) temp. Pollen viability was scored as described in FIG. 1;

FIG. 4 shows bar graphs illustrating relative expression of various sugar metabolizing genes in tomato anther and pollen. LeFRK are fructokinases, LeHXK are hexokinases, TIV1, Lin5-8 are invertases, SuSy are sucrose synthases and LeGLT is a plastidic glucose transporter. Gene expression was determined by real time PCR with gene specific primers and was normalized to cylcophilin as a house keeping gene. Samples with the letter H were taken from plants grown at HTS (32°/26° C.);

FIG. 5 is a bar graph showing the effect of P_{LeFRK4}::AtHXK1 on pollen germination. Control tomato plants (MP) and original (To) independent isogenic transgenic tomato lines overexpressing P_{LeFRK4}::AtHXK1 (assigned PFH lines 1-8) were grown at normal (28° C./22° C.-day/night) temperatures. Pollen were collected and germinated at low (23° C.) and high (32° C.) temp. Pollen viability was scored as described in FIG. 1; and

FIG. 6 shows the nucleotide sequence of the LeFRK4 promoter comprising 2488 bp, followed by the cDNA sequence of the LeFRK4 gene (bp 2489-3821). The promoter was sequenced from BAC colonies using appropriate primers, as indicated below.

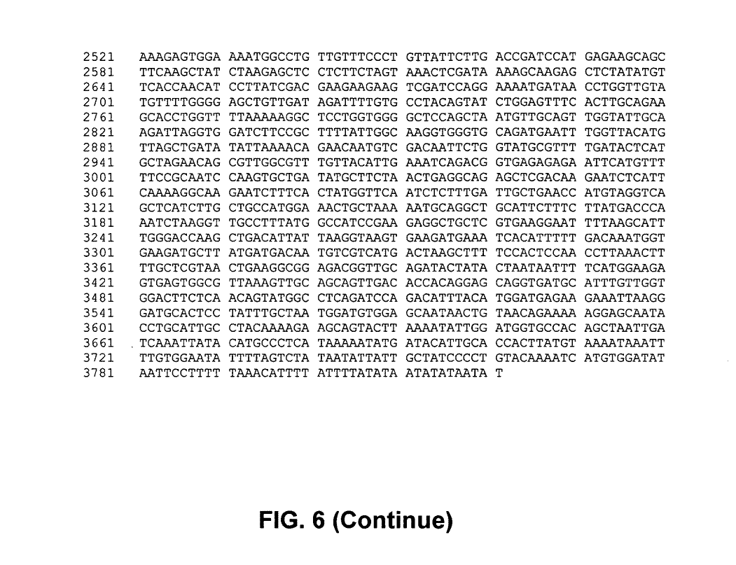

### DETAILED DESCRIPTION OF EXEMPLARY EMBODIMENTS

### List Of Abbreviations

CaMV-Cauliflower mosaic virus

FRK-Fructokinase

Fru6p-Fructose 6-phosphate

Glc6P-Glucose 6-phosphate

HXK-Hexokinase

LeHXK1,2,3,4-L. esculentum hexokinase 1, 2, 3 and 4

LeFRK1,2,3,4-L. esculentum fructokinase 1, 2, 3 and 4

PCR-Polymerase Chain Reaction

SuSy-Sucrose Synthase

UDP-glucose-Uridyl diphosphate glucose

### List of Growth Media

½×MSO: 2.3 gr/lit Murashige and Skoog (MS) salts
3% sucrose
0.8% agar
pH 5.8

Do: 4.3 gr/lit MS salts
3% glucose
1 mg/lit zeatin
0.8% agar
pH 5.8

D1: 4.3 gr/lit MS salts
3% glucose
1 mg/lit zeatin
0.8% agar
100 mg/lit kanamycin
300 mg/lit carbenecelin
pH 5.8

MSR: 4.3 gr/lit MS salts
3% sucrose
1 mg/lit IBA
0.8% agar
pH 5.8

LB: 10 gr/lit tryptone
5 gr/lit yeast extract
5 gr/lit NaCl
15 gr/lit agar

Primers

| | |
|---|---|
| 1. LeHXK1 fw: | |
| GGACTTGCTGGGAGAGGAGT; | (SEQ. ID. NO: 3) |
| 2. LeHXK1 rev: | |
| AAGGTACATTGAATGAGAGGCA; | (SEQ. ID NO: 4) |
| 3. LeHXK2 fw: | |
| GTCCTCCCATCTTCCCTTG; | (SEQ. ID NO: 5) |
| 4. LeHXK2 rev: | |
| CCCAAGTACATACCAGAACAT; | (SEQ. ID NO: 6) |
| 5. LeHXK3 fw: | |
| GCGATATTATCACCTCTCGTG; | (SEQ. ID NO: 7) |
| 6. LeHXK3 rev: | |
| CTGCTTCTCTCCGTCTTTAAA; | (SEQ. ID NO: 8) |
| 7. LeHXK4 fw: | |
| GCTGAGGACACCTGATATATG; | (SEQ. ID NO: 9) |
| 8. LeHXK4 rev: | |
| GATCGGATTTTACCCCAGCTA; | (SEQ. ID NO: 10) |
| 9. LeFRK1 fw: | |
| CTCCGTTACATATCTGATCCT; | (SEQ. ID NO: 11) |
| 10. LeFRK1 rev: | |
| GACAGCATTGAAGTCACCTT; | (SEQ. ID NO: 12) |
| 11. LeFRK2 fw: | |
| TTGTTGGTGCCCTTCTAACCA; | (SEQ. ID NO: 13) |
| 12. LeFRK2 rev: | |
| ACGATGTTTCTATGCTCCTCCCT; | (SEQ. ID NO: 14) |
| 13. LeFRK3 fw: | |
| TTACGGAGCCATGCAAATCAG; | (SEQ. ID NO: 15) |
| 14. LeFRK3 rev: | |
| GCCACAATGGAAGCCCTCAATT; | (SEQ. ID NO: 16) |
| 15. LeFRK4 fw: | |
| GGTGATGCATTTGTTGGTGGAC; | (SEQ. ID NO: 17) |
| 16. LeFRK4 rev: | |
| GCTGTGGCACCATCCAATATTT; | (SEQ. ID NO: 18) |
| 17. TIV1 fw: | |
| GAAGTGGACAAAGTCGCGCTT; | (SEQ. ID NO: 19) |
| 18. TIV1 rev: | |
| CTGGCGTTAGCTCAGATAGCGT; | (SEQ. ID NO: 20) |
| 19. Lin5 fw: | |
| GAACTGAGTTTCGCGATCCAACT; | (SEQ. ID NO: 21) |
| 20. Lin5 rev: | |
| TGAAGTGGATGTTGGGCTTTG; | (SEQ. ID NO: 22) |
| 21. Lin6 fw: | |
| ACCCAAAAGGAGCAACATGG; | (SEQ. ID NO: 23) |
| 22. Lin6 rev: | |
| CCTGGTAAGATTGTGGCTGACC; | (SEQ. ID NO: 24) |
| 23. Lin7 fw: | |
| TTGTTTGGGCTCATTCCGTT; | (SEQ. ID NO: 25) |
| 24. Lin7 rev: | |
| CCGGTGTACAAGATGACTGGCT; | (SEQ. ID NO: 26) |
| 25. Lin8 fw: | |
| AACCCGCAATCTATCCATCCA; | (SEQ. ID NO: 27) |
| 26. Lin8 rev: | |
| TCCGCTGGGATTGCATAGTTT; | (SEQ. ID NO: 28) |
| 27. SuSy1 fw: | |
| TTCACCATGGCAAGATTGGAC; | (SEQ. ID NO: 29) |
| 28. SuSy1 rev: | |
| TCTCTGCCTGCTCTTCCAAGTC; | (SEQ. ID NO: 30) |
| 29. SuSy2 fw: | |
| CATCACCAGCACATTCCAGGA; | (SEQ. ID NO: 31) |
| 30. SuSy2 rev: | |
| AGCTCCAGGTGAGACGATGTTG; | (SEQ. ID NO: 32) |
| 31. LeGT fw: | |
| GGGTTGCTGAGGTTATGAGTGAT | (SEQ. ID NO: 33) |
| T; | |
| 32. LeGT rev: | |
| AGCTGCACCAACGCTAACAA; | (SEQ. ID NO: 34) |
| 33. Cyclo fw: | |
| CGTCGTGTTTGGACAAGTTG; | (SEQ. ID NO: 35) |
| 34. Cyclo rev: | |
| CCGCAGTCAGCAATAACCA; | (SEQ. ID NO: 36) |
| 35. Fk4102: | |
| ATCGAGTTTACTAGAAGAGGA; | (SEQ. ID NO: 37) |
| 36. Fk4128: | |
| GGTGAACATATAGAGCTCTTGCT | (SEQ. ID NO: 38) |
| T; | |
| 37. Fkpro656: | |
| GCATATCAACTCTAAATCACGAA | (SEQ. ID NO: 39) |
| G; | |
| 38. Fkpro659: | |
| GATGCATATCAACTCTAAATCAC | (SEQ. ID NO: 40) |
| G; | |
| 39. Fkpro776: | |
| TGAAGAAGACTAGAGGAATTCCC | (SEQ. ID NO: 41) |
| T; | |
| 40. LeFRK4pro fw: | |
| AGGTTCTAGAGCTCTCCGGAAA; | (SEQ. ID NO: 42) |
| 41. LeFRK4pro rev: | |
| GATCGGTCAAGAATAACAGGG; | (SEQ. ID NO: 43) |
| 42. LeFRK4pro fw: | |
| ACCACTAGATATCCTAAGTAGTA; | (SEQ. ID NO: 44) |
| 43. AtHXK1 fw: | |
| GCGGGAAGCAAGAGCGTGTT; | (SEQ. ID NO: 45) |
| 44. AtHXK1 rev: | |
| CTCCTCGGGTTGCTATGATG; | (SEQ. ID NO: 46) |

### EXAMPLES

### 1. The Effect of HTS on Pollen Development and Germination

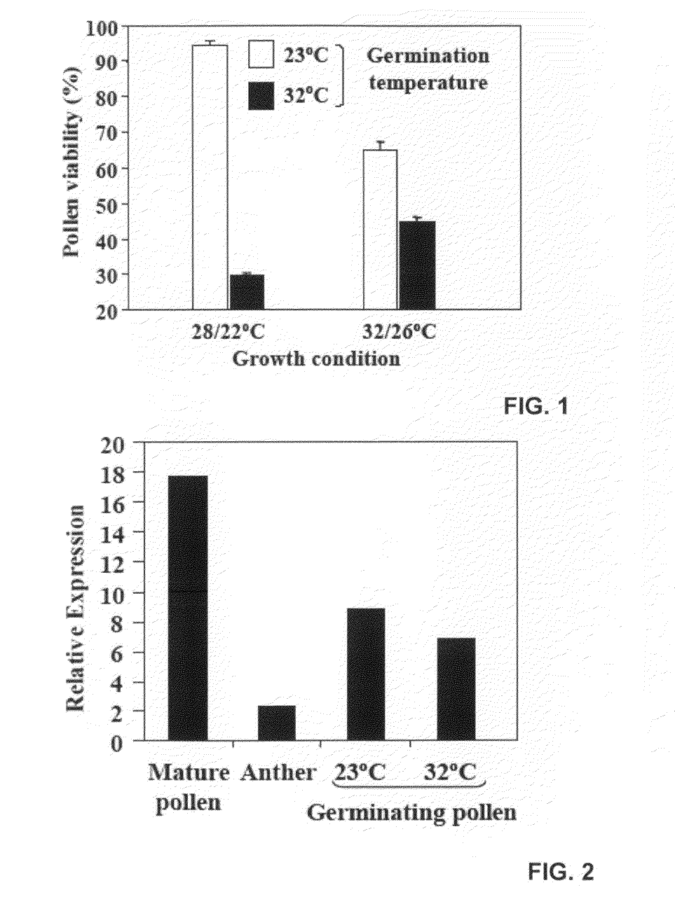

The effect of HTS on both pollen development and germination is demonstrated in FIG. 1. Pollen of tomato plants grown at normal temperatures (28/22° C. day/night) had good viability upon germination at 23° C. and markedly reduced viability upon germination at 32° C., indicating a negative effect of HTS on pollen germination. Pollen of plants grown at high temperatures (32/26° C.) also displayed lower viability when germinated at 23° C. indicating a persisting negative effect of HTS on pollen development.

### 2. Over-Expression of AtHXK1 Improves Development and Germination of Tomato Pollen Under HTS

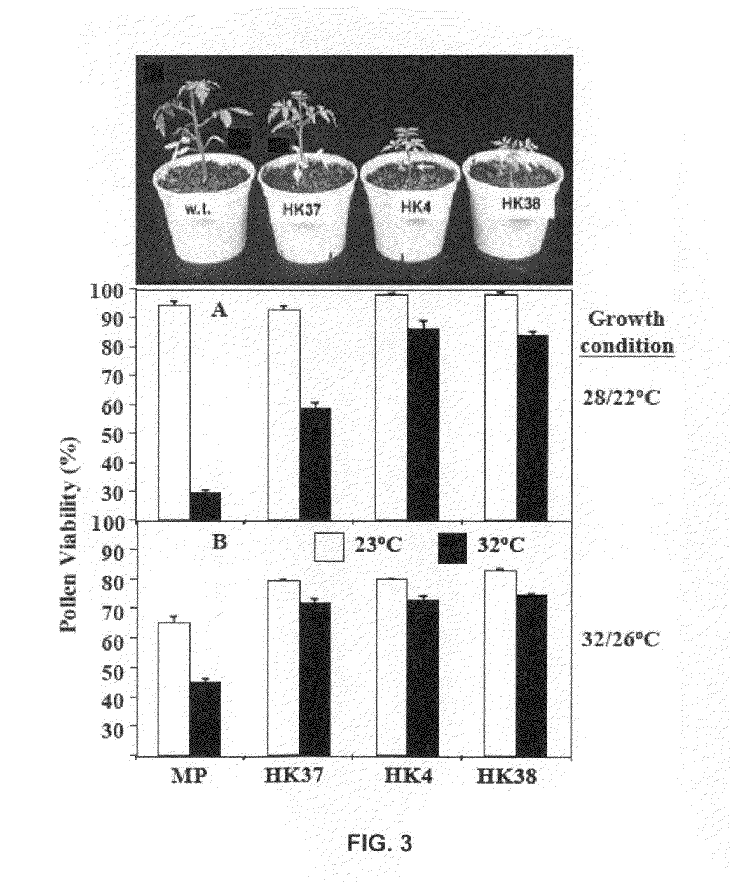

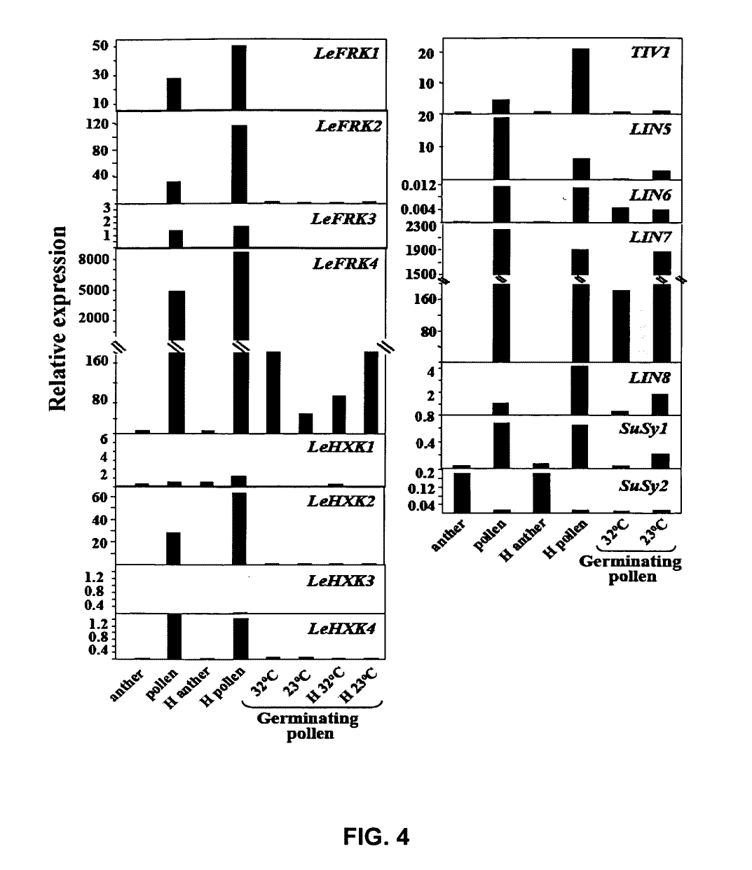

To investigate the importance of hexose phosphorylation in determining pollen competence to germinate, pollen germination was analyzed for tomato plants over-expressing the Arabidopsis hexokinase gene (AtHXK1) under the control of the CaMV 35S promoter (35S::AtHXK1) [8]. Although previous reports claimed that this promoter had only negligible expression in tomato pollen, it was found that the 35S::AtHXK1 construct is expressed in pollen, in accordance with published results for tomato and other plant species [Duck N B, Folk W R (1994) Hsp70 heat shock protein cognate is expressed and stored in developing tomato pollen. Plant Mol Biol 26: 1031-1039], as well as in empty tomato anthers and in pollen germinating at either 23° C. or 32° C. (FIG. 2). Since the CaMV 35S promoter expresses in all plant organs, expression of the Arabidopsis AtHXK1 under this promoter accelerated leaf senescence and inhibited growth [8]. Nevertheless, pollen of tomato plants overexpressing AtHXK1 had a significantly higher viability when either grown or germinated at high (32° C.) temperatures in direct correlation with the occurrence of AtHXK1 expression (FIG. 3). Although HK37, HK4 and HK38 plants have less photosynthetic capacity, they were still able to set fruits upon growth at HTS (32/28° C.) unlike the control plants. These results strongly suggest that hexose phosphorylation in anther and pollen may affect the germination potential of pollen and highlights the need to use anther- and pollen-specific promoters to drive the expression of the AtHXK1.

### 3. LeFRK4 is Highly Expressed in Mature and Germinating Tomato Pollen

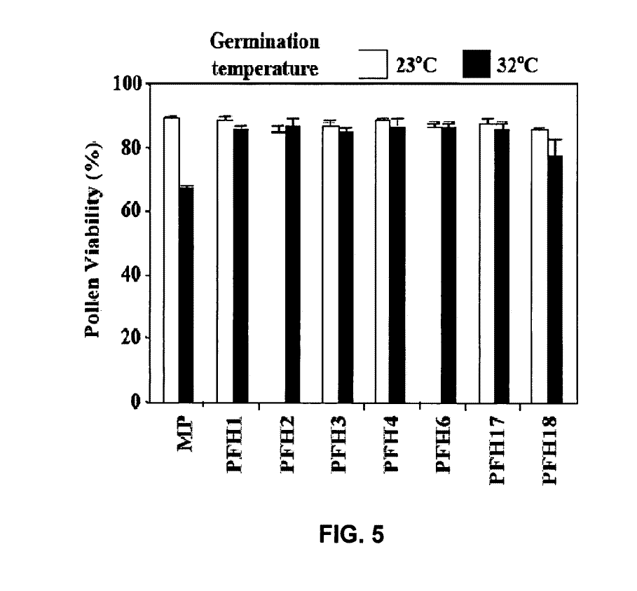

Recently, a fructokinase gene, LeFRK4, was cloned which is exclusively expressed in stamens [6]. The expression of LeFRK4 was analyzed in developing and germinating pollen relative to the other three FRK genes (LeFRK1, 2 & 3), the four known HXK genes (LeHXK1-4), the 5 invertase genes (TIV1, LIN5-8) and the two SuSy genes (SuSy1-2). It was found that LeFRK4 is by far the most abundantly expressed gene in both mature and germinating tomato pollen (FIG. 4). LeFRK4 is also expressed in developing pollen grains and in other parts of the anther, although at a lower level (not shown). The LeFRK4 promoter should therefore be a good candidate to drive anther- and pollen-specific expression.

### 4. Isolation of LeFRK4 Promoter and its Use to Drive Expression of AtHXK1 and LeFRK1

The preferred promoter region of LeFRK4 (SEQ. ID. NO: 2) was isolated and joined to AtHXK1 and to LeFRK1 to obtain P_{LeFRK4}::AtHXK1 and P_{LeFRK4}::LeFRK1 constructs. To address the question of which of the two AtHXK1 activities, glucose or fructose phosphorylation, contributes to HTS resistance, HTS resistance of plants expressing AtHXK1 or LeFRK1 under the control of the LeFRK4 promoter should be compared. To reduce the possibility of cosuppression, LeFRK1 should be used, whose normal expression in anther and pollen is relative low. LeFRK1 should be preferred over LeFRK2 and LeFRK3 since LeFRK1, unlike the latter enzymes (but like LeFRK4), is not repressed by fructose and perhaps could further enhance fructose phosphorylation.

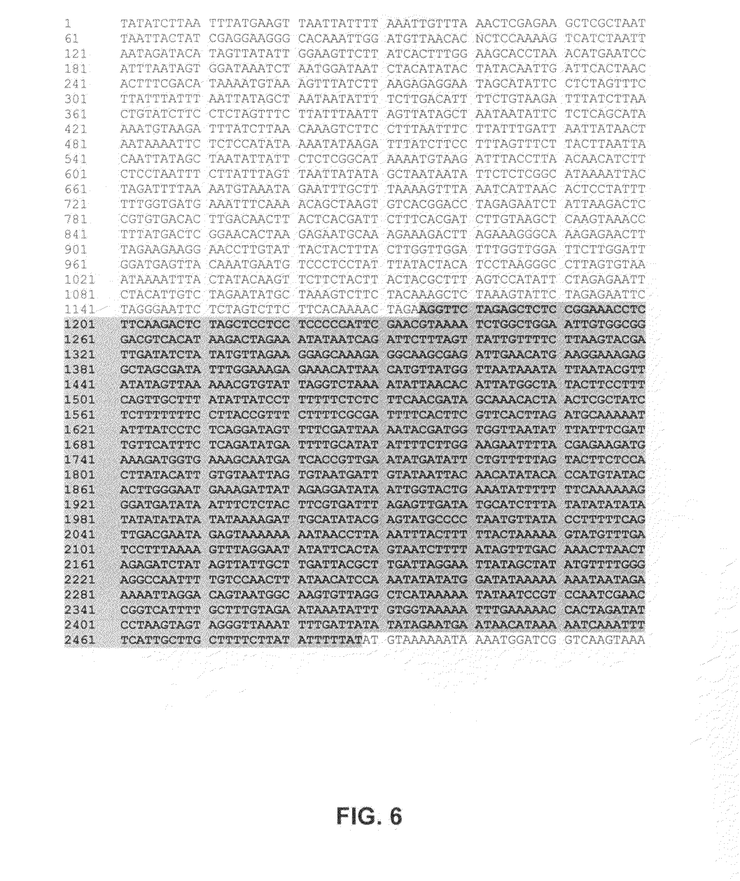

The P_{LeFRK4}::AtHXK1 and P_{LeFRK4}::LeFRK1 constructs have been used to generate about thirty transgenic tomato plants per construct. Initial analysis of To plants expressing P_{LeFRK4}::AtHXK1 has shown that independent transgenic plants exhibit high pollen viability upon germination at high temperature (FIG. 5). In addition, unlike the growth inhibition observed with HK plants (plants expressing AtHXK1 under the CaMV 35S promoter), plants expressing P_{LeFRK4}::AtHXK1 exhibited entirely normal growth and development (not shown).

1. An isolated polynucleotide sequence comprising one of the following:
(a) the LeFRK4 promoter; and
(b) a modified LeFRK4 promoter in which one or more of the nucleotide bases have been substituted or deleted, or one or more nucleotide bases have been added thereto, wherein the specific promoter activity of the modified promoter is substantially the same as that of the LeFRK4 promoter.

2. A vector comprising the polynucleotide sequence of claim 1.

3. A vector according to claim 2 wherein the polynucleotide sequence of claim 1 is operably linked to a heterologous gene.

4. The vector of claim 3 wherein said heterologous gene encodes a protein involved in sugar metabolism.

5. The vector of claim 4 wherein said heterologous gene encodes an invertase, a sucrose synthase, a glucose transporter or a sugar kinase.

6. The vector of claim 5 wherein said sugar kinase is a hexokinase, a fructokinase, a fucokinase or a galactokinase.

7. A host cell comprising the vector of claim 2.

8. A transgenic plant comprising the host cell of claim 7.

9. A method for protecting a plant from high temperature stress (HTS), comprising transforming the plant with a polynucleotide sequence, wherein said polynucleotide sequence comprises a nucleotide sequence encoding a sugar kinase under the control of an anther and/or pollen specific promoter, thereby producing a transformed plant having improved tolerance to HTS.

10. The method of claim 9 wherein said sugar kinase is a hexokinase, a fucokinase, a galactokinase or a fructokinase.

11. The method of claim 9 wherein said anther and/or pollen specific promoter is the LeFRK4 promoter.

12. The method of claim 9 wherein said plant is selected from plants susceptible to HTS.

13. The method of claim 9 wherein said plant is selected from the group consisting of tomato, cucumber, peppers, zucchini, maize, cotton, flax, wheat, rice, eggplant, melon and Brassica.

14. A method for causing male sterility in a plant comprising transforming the plant with a polynucleotide sequence, wherein said polynucleotide sequence comprises a nucleotide sequence capable of disrupting pollen or anther development under the control of an anther and/or pollen specific promoter, thereby causing male sterility.

15. The method of claim 14 wherein said polynucleotide sequence encodes an RNAse, protease or toxin.

16. The method of claim 14 wherein said anther and/or pollen specific promoter is the LeFRK4 promoter.


SEQUENCE LISTING (47 sequences)

SEQ ID NO: 1 - DNA, 2488 bp, Lycopersicon esculentum
  misc_feature (101)..(101): “n” may be either A, or C, or G, or T
tatatcttaatttatgaagttaattattttaaattgtttaaactcgagaagctcgctaattaattactatcgaggaagggcacaaattggatgttaacacnctccaaaagtcatctaattaatagatacatagttatattggaagttcttatcactttggaagcacctaaacatgaatccatttaatagtggataaatctaatggataatctacatatactatacaattgattcactaacactttcgacataaaatgtaaagtttatcttaagagaggaatagcatattcctctagtttcttatttatttaattatagctaataatattttcttgacattttctgtaagatttatcttaactgtatcttcctctagtttcttatttaattagttatagctaataatattctctcagcataaaatgtaagatttatcttaacaaagtcttcctttaatttcttatttgattaattataactaataaaattctctccatataaaatataagatttatcttcctttagtttcttacttaattacaattatagctaatattattctctcggcataaaatgtaagatttaccttaacaacatcttctcctaatttcttatttagttaattatatagctaataatattctctcggcataaaattactagattttaaaatgtaaatagaatttgctttaaaagtttaaatcattaacactcctattttttggtgatgaaatttcaaaacagctaagtgtcacggacctagagaatctattaagactccgtgtgacacttgacaacttactcacgattctttcacgatcttgtaagctcaagtaaacctttatgactcggaacactaagagaatgcaaagaaagacttagaaagggcaaagagaactttagaagaaggaaccttgtattactactttacttggttggatttggttggattcttggattggatgagttacaaatgaatgtccctcctatttatactacatcctaagggccttagtgtaaataaaatttactatacaagttcttctacttactacgctttagtccatattctagagaattctacattgtctagaatatgctaaagtcttctacaaagctctaaagtattctagagaattctagggaattctctagtcttcttcacaaaactagaaggttctagagctctccggaaacctcttcaagactctagctcctcctcccccattcgaacgtaaaatctggctggaattgtggcgggacgtcacataagactagaaatataatcagattctttagttattgttttcttaagtacgattgatatctatatgttagaaggagcaaagaggcaagcgagattgaacatgaaggaaagaggctagcgatatttggaaagagaaacattaacatgttatggttaataaatattaatacgttatatagttaaaaacgtgtattaggtctaaaatattaacacattatggctatacttcctttcagttgctttatattatccttttttctctcttcaacgatagcaaacactaactcgctatctctttttttccttaccgtttcttttcgcgattttcacttcgttcacttagatgcaaaaatatttatcctctcaggatagttttcgattaaaatacgatggtggttaatatttatttcgattgttcatttctcagatatgattttgcatatattttcttggaagaattttacgagaagatgaaagatggtgaaagcaatgatcaccgttgaatatgatattctgtttttagtacttctccacttatacattgtgtaattagtgtaatgattgtataattacaacatatacaccatgtatacacttgggaatgaaagattatagaggatataattggtactgaaatatttttttcaaaaaagggatgatataatttctctacttcgtgatttagagttgatatgcatctttatatatatatatatatatatatataaaagattgcatatacgagtatgcccctaatgttatacctttttcagttgacgaatagagtaaaaaaaataaccttaaatttactttttactaaaaagtatgtttgatcctttaaaagtttaggaatatattcactagtaatcttttatagtttgacaaacttaactagagatctatagttattgcttgattacgcttgattaggaattatagctatatgttttgggaggccaattttgtccaacttataacatccaaatatatatggatataaaaaaaataatagaaaaattaggacagtaatggcaagtgttaggctcataaaaatataatccgtccaatcgaaccggtcattttgctttgtagaataaatatttgtggtaaaaatttgaaaaaccactagatatcctaagtagtagggttaaattttgattatatatagaatgaataacataaaaatcaaattttcattgcttgcttttcttatatttttat

SEQ ID NO: 2 - DNA, 1313 bp, Lycopersicon esculentum
ggttctagagctctccggaaacctcttcaagactctagctcctcctcccccattcgaacgtaaaatctggctggaattgtggcgggacgtcacataagactagaaatataatcagattctttagttattgttttcttaagtacgattgatatctatatgttagaaggagcaaagaggcaagcgagattgaacatgaaggaaagaggctagcgatatttggaaagagaaacattaacatgttatggttaataaatattaatacgttatatagttaaaaacgtgtattaggtctaaaatattaacacattatggctatacttcctttcagttgctttatattatccttttttctctcttcaacgatagcaaacactaactcgctatctctttttttccttaccgtttcttttcgcgattttcacttcgttcacttagatgcaaaaatatttatcctctcaggatagttttcgattaaaatacgatggtggttaatatttatttcgattgttcatttctcagatatgattttgcatatattttcttggaagaattttacgagaagatgaaagatggtgaaagcaatgatcaccgttgaatatgatattctgtttttagtacttctccacttatacattgtgtaattagtgtaatgattgtataattacaacatatacaccatgtatacacttgggaatgaaagattatagaggatataattggtactgaaatatttttttcaaaaaagggatgatataatttctctacttcgtgatttagagttgatatgcatctttatatatatatatatatatatatataaaagattgcatatacgagtatgcccctaatgttatacctttttcagttgacgaatagagtaaaaaaaataaccttaaatttactttttactaaaaagtatgtttgatcctttaaaagtttaggaatatattcactagtaatcttttatagtttgacaaacttaactagagatctatagttattgcttgattacgcttgattaggaattatagctatatgttttgggaggccaattttgtccaacttataacatccaaatatatatggatataaaaaaaataatagaaaaattaggacagtaatggcaagtgttaggctcataaaaatataatccgtccaatcgaaccggtcattttgctttgtagaataaatatttgtggtaaaaatttgaaaaaccactagatatcctaagtagtagggttaaattttgattatatatagaatgaataacataaaaatcaaattttcattgcttgcttttcttatatttttat

SEQ ID NO: 3 - DNA, 20 bp, Artificial Sequence
  LeHXK1 forward primer
ggacttgctgggagaggagt

SEQ ID NO: 4 - DNA, 22 bp, Artificial Sequence
  LeHXK1 reverse primer
aaggtacattgaatgagaggca

SEQ ID NO: 5 - DNA, 19 bp, Artificial Sequence
  LeHXK2 forward primer
gtcctcccatcttcccttg

SEQ ID NO: 6 - DNA, 21 bp, Artificial Sequence
  LeHXK2 reverse primer
cccaagtacataccagaacat

SEQ ID NO: 7 - DNA, 21 bp, Artificial Sequence
  Primer LeHXK3 forward
gcgatattatcacctctcgtg

SEQ ID NO: 8 - DNA, 21 bp, Artificial Sequence
  LeHXK3 reverse primer
ctgcttctctccgtctttaaa

SEQ ID NO: 9 - DNA, 21 bp, Artificial Sequence
  LeHXK4 forward primer
gctgaggacacctgatatatg

SEQ ID NO: 10 - DNA, 21 bp, Artificial Sequence
  LeHXK4 reverse primer
gatcggattttaccccagcta

SEQ ID NO: 11 - DNA, 21 bp, Artificial Sequence
  LeFRK1 forward primer
ctccgttacatatctgatcct

SEQ ID NO: 12 - DNA, 20 bp, Artificial Sequence
  LeFRK1 reverse primer
gacagcattgaagtcacctt

SEQ ID NO: 13 - DNA, 21 bp, Artificial Sequence
  LeFRK2 forward primer
ttgttggtgcccttctaacca

SEQ ID NO: 14 - DNA, 23 bp, Artificial Sequence
  LeFRK2 reverse primer
acgatgtttctatgctcctccct

SEQ ID NO: 15 - DNA, 21 bp, Artificial Sequence
  LeFRK3 forward primer
ttacggagccatgcaaatcag

SEQ ID NO: 16 - DNA, 22 bp, Artificial Sequence
  Primer LeFRK3 reverse
gccacaatggaagccctcaatt

SEQ ID NO: 17 - DNA, 22 bp, Artificial Sequence
  LeFRK4 forward primer
ggtgatgcatttgttggtggac

SEQ ID NO: 18 - DNA, 22 bp, Artificial Sequence
  LeFRK4 reverse primer
gctgtggcaccatccaatattt

SEQ ID NO: 19 - DNA, 21 bp, Artificial Sequence
  TIV1 forward primer
gaagtggacaaagtcgcgctt

SEQ ID NO: 20 - DNA, 22 bp, Artificial Sequence
  TIV1 reverse primer
ctggcgttagctcagatagcgt

SEQ ID NO: 21 - DNA, 23 bp, Artificial Sequence
  Lin5 forward primer
gaactgagtttcgcgatccaact

SEQ ID NO: 22 - DNA, 21 bp, Artificial Sequence
  Lin5 reverse primer
tgaagtggatgttgggctttg

SEQ ID NO: 23 - DNA, 20 bp, Artificial Sequence
  Lin6 forward primer
acccaaaaggagcaacatgg

SEQ ID NO: 24 - DNA, 22 bp, Artificial Sequence
  Lin6 reverse primer
cctggtaagattgtggctgacc

SEQ ID NO: 25 - DNA, 20 bp, Artificial Sequence
  Lin7 forward primer
ttgtttgggctcattccgtt

SEQ ID NO: 26 - DNA, 22 bp, Artificial Sequence
  Lin7 reverse primer
ccggtgtacaagatgactggct

SEQ ID NO: 27 - DNA, 21 bp, Artificial Sequence
  Lin8 forward primer
aacccgcaatctatccatcca

SEQ ID NO: 28 - DNA, 21 bp, Artificial Sequence
  Lin8 reverse primer
tccgctgggattgcatagttt

SEQ ID NO: 29 - DNA, 21 bp, Artificial Sequence
  SuSy1 forward primer
ttcaccatggcaagattggac

SEQ ID NO: 30 - DNA, 22 bp, Artificial Sequence
  SuSy1 reverse primer
tctctgcctgctcttccaagtc

SEQ ID NO: 31 - DNA, 21 bp, Artificial Sequence
  SuSy2 forward primer
catcaccagcacattccagga

SEQ ID NO: 32 - DNA, 22 bp, Artificial Sequence
  SuSy2 reverse primer
agctccaggtgagacgatgttg

SEQ ID NO: 33 - DNA, 24 bp, Artificial Sequence
  LeGT forward primer
gggttgctgaggttatgagtgatt

SEQ ID NO: 34 - DNA, 20 bp, Artificial Sequence
  LeGT reverse primer
agctgcaccaacgctaacaa

SEQ ID NO: 35 - DNA, 20 bp, Artificial Sequence
  Cyclo forward primer
cgtcgtgtttggacaagttg

SEQ ID NO: 36 - DNA, 19 bp, Artificial Sequence
  Cyclo reverse primer
ccgcagtcagcaataacca

SEQ ID NO: 37 - DNA, 21 bp, Artificial Sequence
  Fk4102 primer
atcgagtttactagaagagga

SEQ ID NO: 38 - DNA, 24 bp, Artificial Sequence
  Fk4128 primer
ggtgaacatatagagctcttgctt

SEQ ID NO: 39 - DNA, 24 bp, Artificial Sequence
  Fkpro656 primer
gcatatcaactctaaatcacgaag

SEQ ID NO: 40 - DNA, 24 bp, Artificial Sequence
  Fkpro659 primer
gatgcatatcaactctaaatcacg

SEQ ID NO: 41 - DNA, 24 bp, Artificial Sequence
  Fkpro776 primer
tgaagaagactagaggaattccct

SEQ ID NO: 42 - DNA, 22 bp, Artificial Sequence
  LeFRK4pro forward primer
aggttctagagctctccggaaa

SEQ ID NO: 43 - DNA, 21 bp, Artificial Sequence
  . LeFRK4pro revese primer
gatcggtcaagaataacaggg

SEQ ID NO: 44 - DNA, 23 bp, Artificial Sequence
  LeFRK4pro forward primer
accactagatatcctaagtagta

SEQ ID NO: 45 - DNA, 20 bp, Artificial Sequence
  AtHXK1 forward primer
gcgggaagcaagagcgtgtt

SEQ ID NO: 46 - DNA, 20 bp, Artificial Sequence
  AtHXK1 reverse primer
ctcctcgggttgctatgatg

SEQ ID NO: 47 - DNA, 3821 bp, Lycopersicon esculentum
  misc_feature (101)..(101): “n” may be either A, or C, or T, or G
tatatcttaatttatgaagttaattattttaaattgtttaaactcgagaagctcgctaattaattactatcgaggaagggcacaaattggatgttaacacnctccaaaagtcatctaattaatagatacatagttatattggaagttcttatcactttggaagcacctaaacatgaatccatttaatagtggataaatctaatggataatctacatatactatacaattgattcactaacactttcgacataaaatgtaaagtttatcttaagagaggaatagcatattcctctagtttcttatttatttaattatagctaataatattttcttgacattttctgtaagatttatcttaactgtatcttcctctagtttcttatttaattagttatagctaataatattctctcagcataaaatgtaagatttatcttaacaaagtcttcctttaatttcttatttgattaattataactaataaaattctctccatataaaatataagatttatcttcctttagtttcttacttaattacaattatagctaatattattctctcggcataaaatgtaagatttaccttaacaacatcttctcctaatttcttatttagttaattatatagctaataatattctctcggcataaaattactagattttaaaatgtaaatagaatttgctttaaaagtttaaatcattaacactcctattttttggtgatgaaatttcaaaacagctaagtgtcacggacctagagaatctattaagactccgtgtgacacttgacaacttactcacgattctttcacgatcttgtaagctcaagtaaacctttatgactcggaacactaagagaatgcaaagaaagacttagaaagggcaaagagaactttagaagaaggaaccttgtattactactttacttggttggatttggttggattcttggattggatgagttacaaatgaatgtccctcctatttatactacatcctaagggccttagtgtaaataaaatttactatacaagttcttctacttactacgctttagtccatattctagagaattctacattgtctagaatatgctaaagtcttctacaaagctctaaagtattctagagaattctagggaattctctagtcttcttcacaaaactagaaggttctagagctctccggaaacctcttcaagactctagctcctcctcccccattcgaacgtaaaatctggctggaattgtggcgggacgtcacataagactagaaatataatcagattctttagttattgttttcttaagtacgattgatatctatatgttagaaggagcaaagaggcaagcgagattgaacatgaaggaaagaggctagcgatatttggaaagagaaacattaacatgttatggttaataaatattaatacgttatatagttaaaaacgtgtattaggtctaaaatattaacacattatggctatacttcctttcagttgctttatattatccttttttctctcttcaacgatagcaaacactaactcgctatctctttttttccttaccgtttcttttcgcgattttcacttcgttcacttagatgcaaaaatatttatcctctcaggatagttttcgattaaaatacgatggtggttaatatttatttcgattgttcatttctcagatatgattttgcatatattttcttggaagaattttacgagaagatgaaagatggtgaaagcaatgatcaccgttgaatatgatattctgtttttagtacttctccacttatacattgtgtaattagtgtaatgattgtataattacaacatatacaccatgtatacacttgggaatgaaagattatagaggatataattggtactgaaatatttttttcaaaaaagggatgatataatttctctacttcgtgatttagagttgatatgcatctttatatatatatatatatatatatataaaagattgcatatacgagtatgcccctaatgttatacctttttcagttgacgaatagagtaaaaaaaataaccttaaatttactttttactaaaaagtatgtttgatcctttaaaagtttaggaatatattcactagtaatcttttatagtttgacaaacttaactagagatctatagttattgcttgattacgcttgattaggaattatagctatatgttttgggaggccaattttgtccaacttataacatccaaatatatatggatataaaaaaaataatagaaaaattaggacagtaatggcaagtgttaggctcataaaaatataatccgtccaatcgaaccggtcattttgctttgtagaataaatatttgtggtaaaaatttgaaaaaccactagatatcctaagtagtagggttaaattttgattatatatagaatgaataacataaaaatcaaattttcattgcttgcttttcttatatttttatatgtaaaaaataaaatggatcggtcaagtaaaaaagagtggaaaatggcctgttgtttccctgttattcttgaccgatccatgagaagcagcttcaagctatctaagagctcctcttctagtaaactcgataaaagcaagagctctatatgttcaccaacatccttatcgacgaagaagaagtcgatccaggaaaatgataacctggttgtatgttttggggagctgttgatagattttgtgcctacagtatctggagtttcacttgcagaagcacctggttttaaaaaggctcctggtggggctccagctaatgttgcagttggtattgcaagattaggtggatcttccgcttttattggcaaggtgggtgcagatgaatttggttacatgttagctgatatattaaaacagaacaatgtcgacaattctggtatgcgttttgatactcatgctagaacagcgttggcgtttgttacattgaaatcagacggtgagagagaattcatgtttttccgcaatccaagtgctgatatgcttctaactgaggcagagctcgacaagaatctcattcaaaaggcaagaatctttcactatggttcaatctctttgattgctgaaccatgtaggtcagctcatcttgctgccatggaaactgctaaaaatgcaggctgcattctttcttatgacccaaatctaaggttgcctttatggccatccgaagaggctgctcgtgaaggaattttaagcatttgggaccaagctgacattattaaggtaagtgaagatgaaatcacatttttgacaaatggtgaagatgcttatgatgacaatgtcgtcatgactaagcttttccactccaaccttaaacttttgctcgtaactgaaggcggagacggttgcagatactatactaataattttcatggaagagtgagtggcgttaaagttgcagcagttgacaccacaggagcaggtgatgcatttgttggtggacttctcaacagtatggcctcagatccagacatttacatggatgagaagaaattaagggatgcactcctatttgctaatggatgtggagcaataactgtaacagaaaaaggagcaatacctgcattgcctacaaaagaagcagtacttaaaatattggatggtgccacagctaattgatcaaattatacatgccctcataaaaatatgatacattgcaccacttatgtaaaataaattttgtggaatattttagtctataatattattgctatcccctgtacaaaatcatgtggatataattcctttttaaacattttattttatataatatataatat